(19) United States
(12) Patent Application Publication
(10) Pub. No.: US 2004/0092970 A1
(43) Pub. Date: May 13, 2004

(54) Prosthetic mesh anchor device

(76) Inventor: Alfredo F. Xavier, Mattapoisett, MA (US)

Correspondence Address: Chester E. Flavin, Ross, Ross & Flavin, Suite 207, 120 Maple Street, Springfield, MA, 01103, US

(21) Appl. No.: 10/272,405

(22) Filed: Oct. 18, 2002

(51) Int. Cl. A61B 17/08

(52) U.S. Cl. 606/151

(57) ABSTRACT

A percutaneous mesh anchoring and placement device for use in groin hernia repair wherein trocars are positioned along a patient's abdominal midline and extend into an insufflated pre-peritoneal space, the device being introduced into the pre-peritoneal space through a selected one of the trocars and comprising a tubular sleeve which encases a spirally-wound roll of prosthetic mesh having an anchor captured therein, the anchor having a string attached thereto which extends outwardly through provided orifices in the prosthetic mesh and sleeve whereby the string may be grasped by the surgeon to position and hold the device firmly against the posterior abdominal wall, the sleeve being separable from the mesh and removable from the pre-peritoneal space through a selected one of the trocars, the orifice in the mesh being of suitable size as to permit the passage of a selected one of the trocars therethrough, whereby the prosthetic mesh may be partially unfurled and an edge stapled to the abdominal wall, the anchor being separable from the mesh and removable from the pre-peritoneal space through a selected one of the trocars, whereby the prosthetic mesh may be fully unfurled and its' remaining edges stapled to the abdominal wall to repair the hernia.

### BACKGROUND OF THE INVENTION

1. Field of the Invention

The invention relates to the laparoscopic repair of groin hernias.

2. Description of Related Art

Laparoscopic repair of groin hernias, including inguinal and femoral hernias, using prosthetic mesh is gaining increasing popularity among surgeons. It offers comparable results to conventional open repair, with significantly less pain and disability. Despite its solid anatomical and physiological principles, as well as excellent patient acceptance, it still offers significant technical challenges to the average surgeon. Specifically, proper anatomical placement of the mesh, sufficient coverage of all anatomically weak areas, and the avoidance of wrinkles or folds, constitute the most crucial determinants for a successful procedure, leading to decreased post-operative pain and reduced recurrence rate. These important features are among the objects of this invention.

To the best of our knowledge, there is currently no system devised to secure mesh placement for laparoscopic groin hernia repair using a percutaneous anchoring device. The current standard technique uses a rectangular mesh usually rolled into a cylinder, introduced through a trocar and dropped into the pre-peritoneal space.

The mesh is then unfolded by manipulation with two laparoscopic forceps and placed against the anterior abdominal wall, where it is stapled into place. During this process, in most instances, the mesh is entirely loose inside the pre-peritoneal space and its proper placement depends wholly on the surgeon's ability and quick reflexes. In order to facilitate this difficult process of free manipulation, several mesh designs are available, addressing the issues of conformability, porosity and elasticity. All available methods to facilitate proper placement of the mesh only address the issues of mesh design and different means to deploy and secure the mesh by instrumental manipulation within the pre-peritoneal space

### BRIEF SUMMARY OF THE INVENTION

The invention hereof addresses this singular problem by providing a simple and quick percutaneous anchoring system for the mesh, which allows its proper anatomical placement against the abdominal wall, thereby avoiding the difficult process of free manipulation and greatly facilitating the technical procedure while reducing operative time and cost.

The prosthetic mesh anchor device of the invention includes a tubular, cylindrical sleeve having a closed end and an open end, the sleeve encasing a spirally-wound mesh roll and an anchor which has a string secured thereto, the string extending outwardly through provided orifices in the mesh and the sleeve.

The current invention consists of a unitary basic unit which includes an anchoring system for the mesh, enabling prompt and accurate placement of the mesh in a single step. An anchor firmly secures the mesh in a stationary position in the exact desired anatomical location, allowing the surgeon to staple the mesh against the abdominal wall, while it remains secured in the correct position. The device eliminates the difficult manipulation process that is characteristic of the currently available techniques and mesh designs. Once correct position and vertical orientation of the medial portion of the mesh is secured, the surgeon assumes total control of the mesh and the remaining steps of the procedure become considerably easier and faster.

The procedure utilizing the prosthetic mesh anchor device hereof consists of the following steps:

Step 1. Creation of the Pre-Peritoneal Space

A small incision is made below the umbilicus, slightly toward the side of the hernia. The fascia is opened and a peritoneal distention balloon trocar is introduced into the pre-peritoneal space and tunneled toward the pubis. A laparoscope is introduced and the balloon is then gradually inflated. This process is monitored both visually and manually by the surgeon to a volume that provides satisfactory exposure of the anatomical structures. Once the space is created, the balloon trocar is removed and replaced with a blunt sealing infra-umbilical trocar and the laparoscope reinserted.

The pre-peritoneal space is reinsufflated to a pressure of 8 to 12 mm of Hg. Two additional trocars are then inserted along the midline, under direct vision, namely, a 5 mm supra-pubic trocar which is inserted just above the pubis, and another 5 mm or 10 mm middle trocar which is inserted halfway between the pubis and the umbilicus.

Step 2. Dissection of the Hernia Sac and Spermatic Cord.

Once the pre-peritoneal space is under direct vision, a broader dissection of the pre-peritoneal space is undertaken with two blunt forceps, with a two handed technique, in order to identify the anatomical landmarks and to create sufficient space for placement of the mesh. Cooper's ligament, the inferior epigastric vessels, the pubis and the femoral vessels are identified. In the case of a direct hernia, the sac and pre-peritoneal contents are dissected and separated from the inguinal floor.

In the case of an indirect inguinal hernia, the internal ring is identified with the cord elements. The sac is then reduced and separated from the cord structures.

The cord structures are freed from their attachments and suspended from the posterior abdominal wall. In the case of femoral hernia repair, a similar dissection is performed.

Step 3. Placement of the Mesh.

A blunt forceps is introduced through the supra-pubic trocar and advanced toward the infra-umbilical trocar. Under direct laparoscopic vision, the forceps is then inserted into the infra-umbilical trocar in a retrograde fashion and advanced until it exits the proximal end of the trocar, as the laparoscope is gradually pulled out.

The mesh-anchor device of the invention and its string are brought to the field and the string is grasped. The forceps is then pulled back into the preperitoneal space. and out of the supra-pubic trocar bringing the string out with it.

The surgeon will then hold and pull the string out gradually as the mesh anchor device is introduced through the infra-umbilical trocar and advanced towards the pubis.

As the mesh anchor device reaches the lowest level of penetration, additional tension is applied to the string, bringing the mesh anchor device tight against the abdominal wall. The surgeon will manipulate the sleeve in such a way that the mesh orifice will be placed just beneath the trocar site, in a two-handed coordinated maneuver. At the completion of this maneuver, the string will assume a perpendicular position in relation to the anchor.

When this position is secured, the cylindrical sleeve is pulled out of the pre-peritoneal space by simple traction, leaving behind the anchor and furled mesh held tightly against the abdominal wall by tension exerted on the string by the surgeon.

A laparoscope is reintroduced through the infra-umbilical trocar and the anatomical position and orientation of the mesh are adjusted if necessary.

The medial edge of the mesh assumes a vertical orientation and, once the mesh is stabilized by traction obtained by continued tension exerted on the string, the mesh is partially unrolled laterally by the forceps inserted through the middle trocar. The medial portion of the mesh is then stapled alongside the anchor, providing the desired stability that allows completion of the procedure.

Step 4. Removal of the Anchor.

Once the mesh is placed and stapled along its medial, vertical edge, the anchor is removed. A forceps is introduced through the middle trocar, under direct vision, and the superior edge of the anchor is grasped and pulled out of the pre-peritoneal space, bringing along the string. Alternatively, the string can also be cut with laparoscopic scissors and pulled out through the supra pubic trocar.

Once the anchor has been removed, the surgeon can resume unrolling and placing the mesh with bi-manual control whereupon the remaining edges of the mesh may be stapled to the abdominal wall. Two laparoscopic forceps are re-introduced through the middle and supra-pubic trocars. The circular orifice of the mesh will allow insertion of the supra-pubic trocar into the pre-peritoneal space.

It should be noted that the mesh orifice is much larger than the 5 mm trocar orifice. This will provide two advantages: Just before placing the first staples on the medial vertical side of the mesh, the surgeon has some “wiggle room”, loosening the anchor and making some final adjustment to the mesh, before stapling, in addition, the larger size mesh orifice facilitates reinsertion of the suprapubic trocar after the initial stapling of the mesh. The mesh orifice can be actually fairly large, without compromising the effectiveness of the system.

All trocars are removed from the abdomen and the pre-peritoneal space is deflated.

The wounds are closed.

The open or distal end of the tubular sleeve of the mesh anchor device hereof has a tapered, curvilinear configuration. This is an anatomical design which allows the surgeon to reach down behind the pubic bone, with the end of the sleeve conforming to the curvilinear shape of the bone. When the surgeon inserts the sleeve/mesh assembly, he does it blindly. The curvilinear tip of the sleeve will help him sense the deepest portion of the cavity, as the sleeve falls into the anatomical slot.

With the mesh anchor device hereof, the anchor is disposed immediately below the outer convolution of the mesh roll, which in turn is encased by the sleeve, with the string being fixed at one end to the anchor and having a free end which extends outwardly through aligned orifices in the mesh and the sleeve where it may be grasped by the surgeon using forceps or the like.

In the preferred embodiment of the anchor, a centrally-located tab is provided to which one end of the string is attached.

In a first modified form of anchor, a pair of vertically-spaced, centrally-located orifices are provided, with one end of the string being threaded through one orifice and brought back on itself through the other orifice and knotted.

In a second modified form of anchor, a pair of vertically-spaced, centrally-located diagonal slots are provided, with one end of the string being threaded through one slot and brought back on itself through the other slot and knotted.

In the modified forms of anchor, the centrally-located orifices and/or slots will also be positioned at the center of the mesh orifice as the sleeve is placed against the abdominal wall while being pulled by the string.

The trocar perforation through the abdominal wall will coincide with the mesh orifice and the central orifices and slots of the modified forms of anchor. Once the anchor is withdrawn, the mesh orifice will be co-axial with the trocar opening and the laparoscopic trocar can be re-inserted through the abdominal wall and through the mesh orifice.

In all anchor embodiments, the mesh extends beyond both the upper and lower ends of the anchor. The vertical edges of the mesh also extend beyond the longitudinal edges of the anchor. This configuration provides sufficient mesh exposure in a safe anatomical area for placement of staples through the mesh against the abdominal wall.

The anchors have a semicircumferential cross-section with a smaller diameter than a 5 mm trocar. This is a distinct advantage, since the anchor must be removed through such a laparoscopic trocar.

### BRIEF DESCRIPTION OF THE SEVERAL VIEWS OF THE DRAWINGS

FIG. 1 is a perspective view of a prosthetic mesh anchor device embodying a preferred form of the invention;

FIG. 2 is a perspective view of the sleeve of the mesh anchor device of FIG. 1;

FIG. 2A is an end elevational view of the sleeve of FIG. 2 as seen from the left;

FIG. 3 is a top plan view of the anchor of the mesh anchor device of FIG. 1;

FIG. 3A is an end elevational view of the anchor of FIG. 3;

FIG. 4 is a cross-sectional view taken on line 4-4 of FIG. 3;

FIG. 5 is a top plan view of a first modified form of anchor;

FIG. 5A is a top plan view of a second modified form of anchor;

FIG. 6 is a top perspective view of the surgical mesh roll of the mesh anchor device of FIG. 1;

FIG. 7 is a cross-sectional view showing a preperitoneal space following insufflation and the placement of an upper or infra-umbilical trocar, a middle trocar and a lower or supra-pubic trocar preparatory to use of the mesh anchor device of FIG. 1 for laparoscopic groin hernia repair;

FIG. 8 is a cross-sectional view similar to FIG. 6 showing the mesh anchor device of the invention following its advancement through the infra-umbilical trocar into the preperitoneal space, where it is grasped by a forceps or the like which have been inserted through the supra-pubic trocar;

FIG. 9 is an enlarged, fragmentary, cross-sectional view of the mesh anchor device of the invention following its advancement through the preperitoneal space to a position wherein the innermost end of its sleeve is disposed behind the pubic bone and its string has been placed under tension so as to be disposed perpendicular to the sleeve, with its outer free end disposed outside the abdomen, the middle trocar having been omitted for clarity of illustration;

FIG. 10 is an enlarged, fragmentary, cross-sectional view of the mesh anchor device of the invention following partial removal of its sleeve from the preperitoneal space through the infra-umbilical trocar, with its anchor and furled mesh roll being held firmly against the inner wall of the abdomen by tension placed on its string, the middle trocar having been omitted for clarity of illustration;

FIG. 11 is a fragmentary front elevational view of the mesh roll and anchor of the mesh anchor device of the invention shown in solid lines before the mesh is fully unrolled and shown in dash lines showing the final positioning of the mesh after it is fully unrolled to cover inguinal structures;

FIG. 12 is a cross-sectional view of the mesh anchor device of the invention following stapling of the mesh, with tension on the string having been released so that the anchor can be grasped by forceps and removed from the preperitoneal space through the middle trocar, bringing the string with it;

FIG. 13 is a transverse cross-sectional view of the mesh anchor device of the invention taken at the level of the supra-pubic trocar site, with the string of the device anchored at its lower end to the anchor of FIG. 3 and having its upper free end disposed outwardly of the abdomen; and

FIG. 14 is a transverse cross-sectional view similar to FIG. 12 taken at the level of the supra-pubic trocar site following removal of the sleeve, with the anterior portion of the mesh disposed between the anchor and abdominal wall, the string passing through a mesh orifice and the supra-pubic trocar orifice, and a loop of the string encircling the inferior portion of the tab of the anchor.

### DETAILED DESCRIPTION OF THE INVENTION

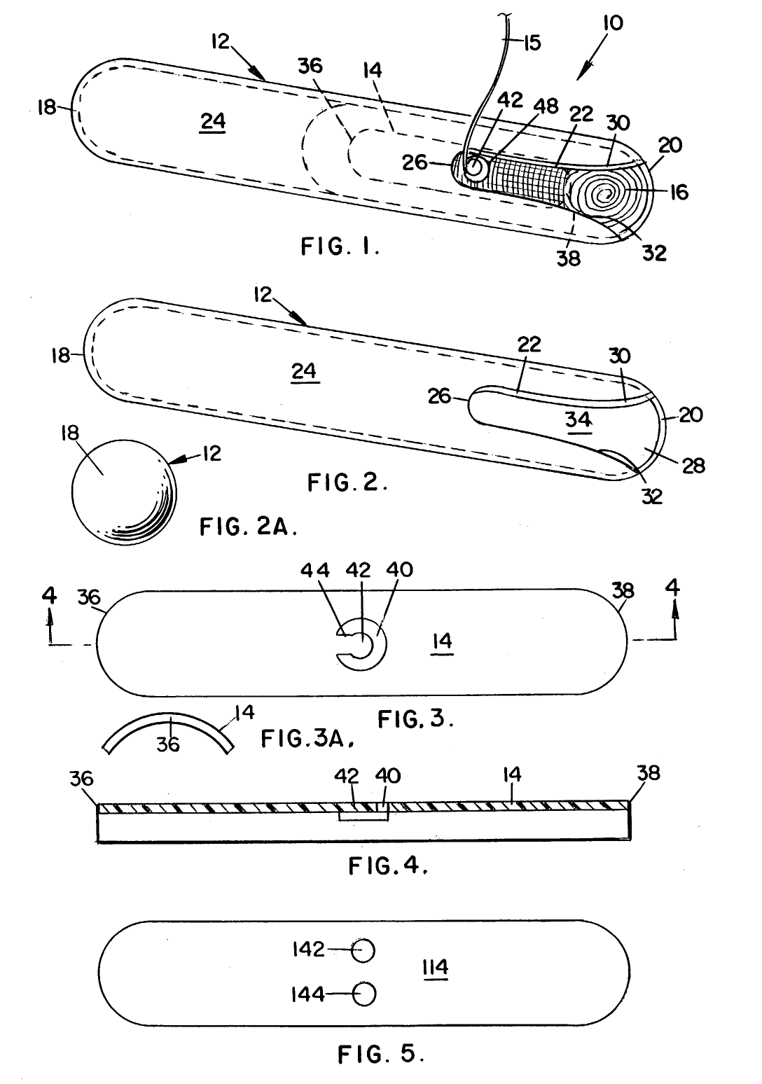

Referring to FIGS. 1-4, a prosthetic mesh anchor device embodying a preferred form of the invention is generally indicated by 10 and includes a hollow, tubular, cylindrical sleeve 12, which encases an anchor 14 having a string 15 attached thereto, and a spirally-wound, cylindrical roll of prosthetic mesh 16.

Sleeve 12 and anchor 14 are preferably fabricated from a thermoplastic material such as polycarbonate or the like.

Sleeve 12 is hollow throughout its length and has a rounded, semicircular, closed end 18 and an open end or tip 20, with end 18 being closed to avoid gas leakage during use.

A somewhat rectangular slot 22 is provided in an upper wall 24 of sleeve 12 and extends longitudinally inwardly from open end 20 along the central axis of the sleeve for approximately one-quarter the length of the sleeve.

Slot 22 has a closed inner end 26 and an opposite open outer end 28.

Slot inner end 26 is of semi-circular configuration, while slot outer end 28 is of tapered, curvilinear configuration wherein the outer ends of the slot curve outwardly as at 30 and 32 from upper wall 24 and merge at their lower ends with a lower wall 34 of sleeve 12, thereby imparting a curvilinear configuration to the open end or tip 20 of the sleeve, all for purposes to appear.

Anchor 14, best seen in FIGS. 1, 3, 3A and 4, is relatively thin in thickness and narrow in width, is of substantially rectangular configuration in plan, has a semi-circumferential transverse cross-section and has curved opposite ends 36 and 38.

A crescent shaped opening 40 cut centrally through anchor 14, provides an integral tab 42 which is joined to the body of the anchor by a neck 44 of reduced width, whereby an end of string 15, not shown, may be looped there-around and knotted so as to be readily secured to the anchor.

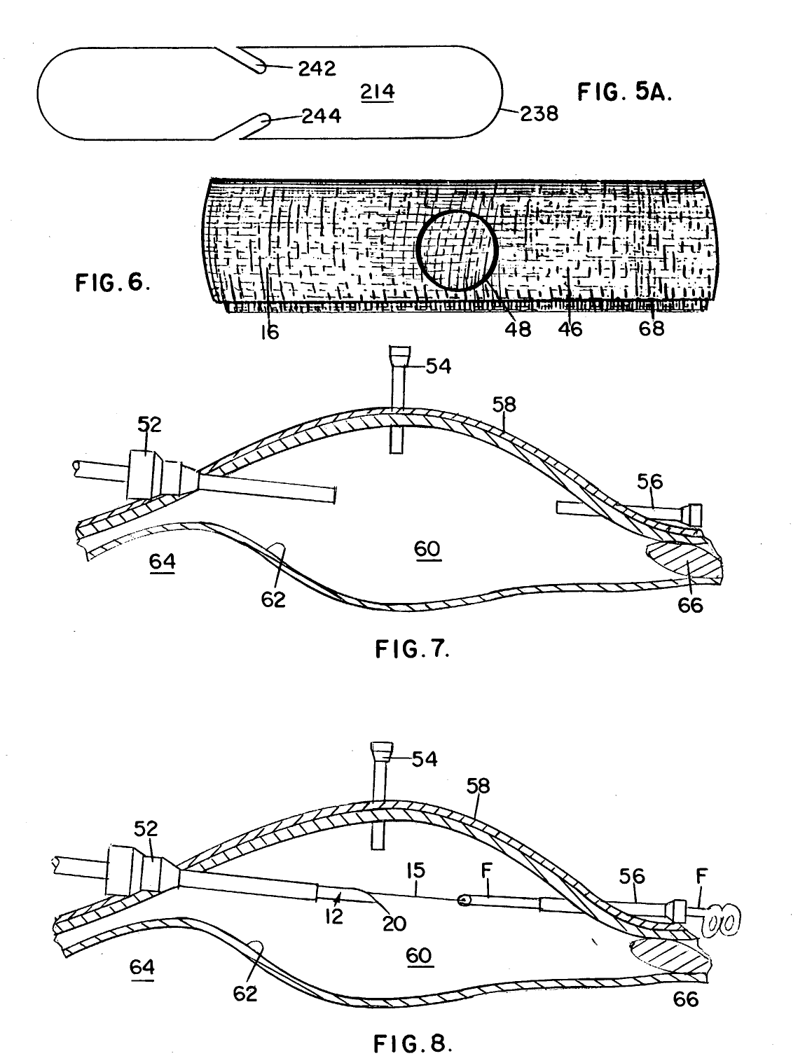

A first modified form of anchor 114, as shown in FIG. 5, is identical to anchor 14 except that tab 42 of anchor 14 has been replaced by a pair of vertically spaced openings 142 and 144 located on the central vertical axis of the anchor.

Anchor 114 is preferably fabricated from a thermoplastic material such as polycarbonate or the like.

In the case of anchor 114, one end of string 15, not shown, may be threaded through one of the openings 142 or 144 and brought back on itself through the other opening and knotted to the string, whereby the string is secured to the anchor.

A second modified form of anchor 214, as shown in FIG. 5A, is identical to anchor 14 except that tab 42 of anchor 14 has been replaced by a pair of oppositely inclined, diagonally directed slots 242 and 244 located on the central vertical axis of the anchor, with the slots extending inwardly from opposite side edges of anchor 214, and the direction of inclination of the slots being toward a forward edge 238 of anchor 214.

Anchor 214 is preferably fabricated from a thermoplastic material such as polycarbonate or the like.

In the case of anchor 214, one end of string 15, not shown, may be threaded through one of the slots 242 or 244 and brought back on itself through the other slot and knotted to the string, whereby the string is secured to the anchor.

Spirally-wound prosthetic mesh roll 16 is approximately one-third the length of sleeve 12 and is approximately one inch greater in length than anchors 14, 114 or 214.

Anchors 14, 114 or 214, are disposed centrally of the length of mesh roll 16 along the longitudinal central axis of the mesh roll immediately below an outer convolution 46 of the mesh roll.

Outer convolution 46 of mesh roll 16 is provided with a central circular orifice 48 so positioned relative to tab 42 of anchor 14, or relative to openings 142 and 144 of anchor 114, or relative to slots 242 and 244 of anchor 214, as to permit a free end of string 15 to pass there through.

The transverse widths of each of anchors 14, or 114, or 214 correspond to approximately one-half the diameter of mesh roll 16, for purposes to appear.

As best seen in FIG. 11, mesh roll 16 is provided with a horizontally inwardly-extending lateral slit 50 to accommodate a trio of cord structures 74 of the patient adjacent the hernia site, as will appear.

Referring to FIGS. 7-14, the manner of use of mesh anchor device 10 will be explained.

In FIG. 7, a 5 mm infra-umbilical trocar 52, a 5 mm or 10 mm middle trocar 54 and a 5 mm supra-pubic trocar 56 have been inserted along the midline of the patient's abdomen 58 following insufflation of a preperitoneal space 60 above the peritoneum 62 and abdominal cavity 64 and adjacent pubis 66 in preparation for laparoscopic hernia repair, with infra-umbilical trocar 52 positioned below the umbilicus and slightly toward the side of the hernia, middle trocar 54 positioned half-way between the pubis and umbilicus, and supra-pubic trocar 56 positioned immediately above the pubis.

Following dissection of the hernia sac and spermatic cord, the surgeon introduces mesh anchor device 10 into preperitoneal space 60, as seen in FIG. 8, with a forceps F advanced through supra-pubic trocar 56 grasping string 15 and drawing mesh anchor device 10 through infra-umbilical trocar 52 with the open end 20 of sleeve 12 of the mesh anchor device facing the supra-pubic trocar.

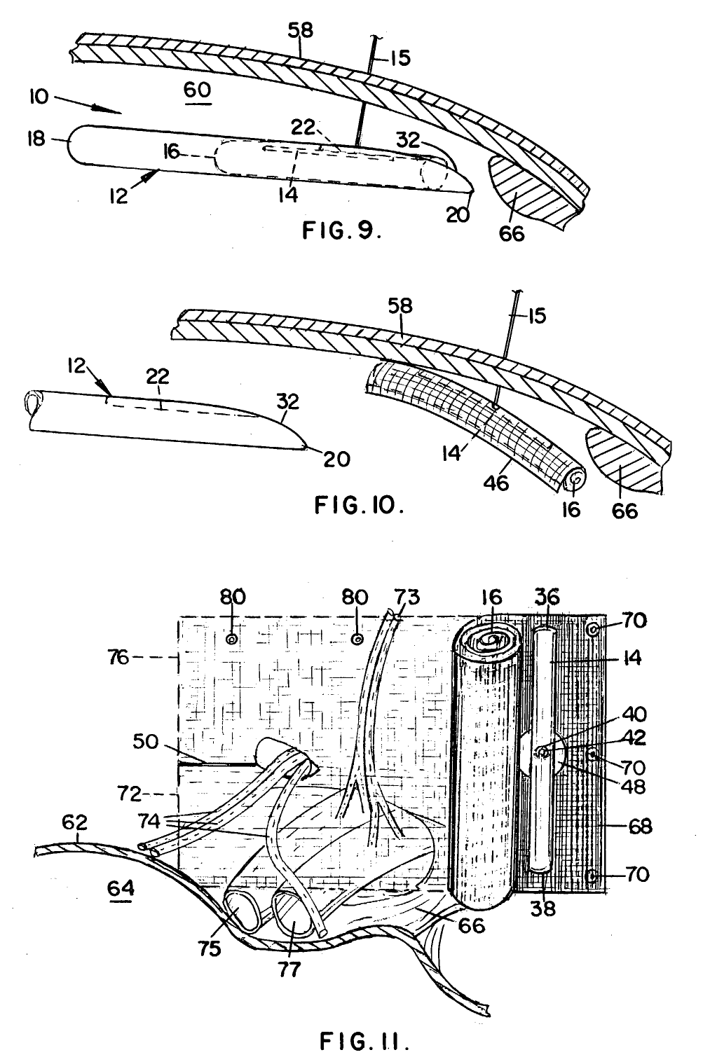

As shown in FIG. 9, the surgeon continues to pull string 15 until open end 20 of sleeve 12 is brought into contact with pubis 66.

As mesh anchor device 10 reaches the lowest level of penetration, additional tension is applied to string 15, bringing the mesh anchor device tight against the abdominal wall as shown in FIG. 10. The surgeon will manipulate sleeve 12 in such a way that orifice 48 of mesh roll 16 is placed just beneath the site of supra-pubic trocar 56 in a two handed maneuver. At this time, string 15 will have assumed a perpendicular position in relation to sleeve 12.

When this position is secured, cylindrical sleeve 12 is pulled out of pre-peritoneal space 60 through infra-umbilical trocar 52 by simple traction, leaving behind anchor 14, or 114, or 214, with furled mesh 16 held tightly against the abdominal wall 58 by tension exerted by the surgeon on string 15.

At this time, a laparoscope is reintroduced through infra-umbilical trocar 52 and the anatomical position and orientation of mesh roll 16 are adjusted, if necessary.

As seen in FIG. 11, a medial edge portion 68 of mesh roll 16 assumes a vertical orientation and, once the mesh is stabilized by traction exerted through string 15, it is partially unrolled laterally by forceps, not shown, inserted through middle trocar 54. Medial edge portion 68 of the mesh roll is then stapled adjacent anchor 14 to the posterior abdominal wall as by staples 70, providing desired stability to allow completion of the procedure.

It should be noted that the upper and lower edges of mesh roll 16 extend beyond both the upper and lower ends 36 and 38 respectively of anchor 14. The vertical edges of the mesh also extend beyond the longitudinal edges of the anchor. This configuration provides sufficient mesh exposure in a safe anatomical area for placement of staples through the mesh against the abdominal wall.

As shown in dash lines in FIG. 11, a superior portion 76 of mesh roll 16 is passed over a pair of inferior epigastric vessels 73, while an inferior portion 72 of mesh roll 16 is passed under cord structures 74, a femoral artery 75 and a femoral vein 77, with the mesh being positioned according to the surgeon's preference.

As aforesaid, horizontal lateral slit 50 in mesh 16 accommodates the cord structures 74.

The upper and lower horizontal edges of the mesh are secured against the posterior abdominal wall by staples 80 as the placement proceeds according to the surgeon's preference.

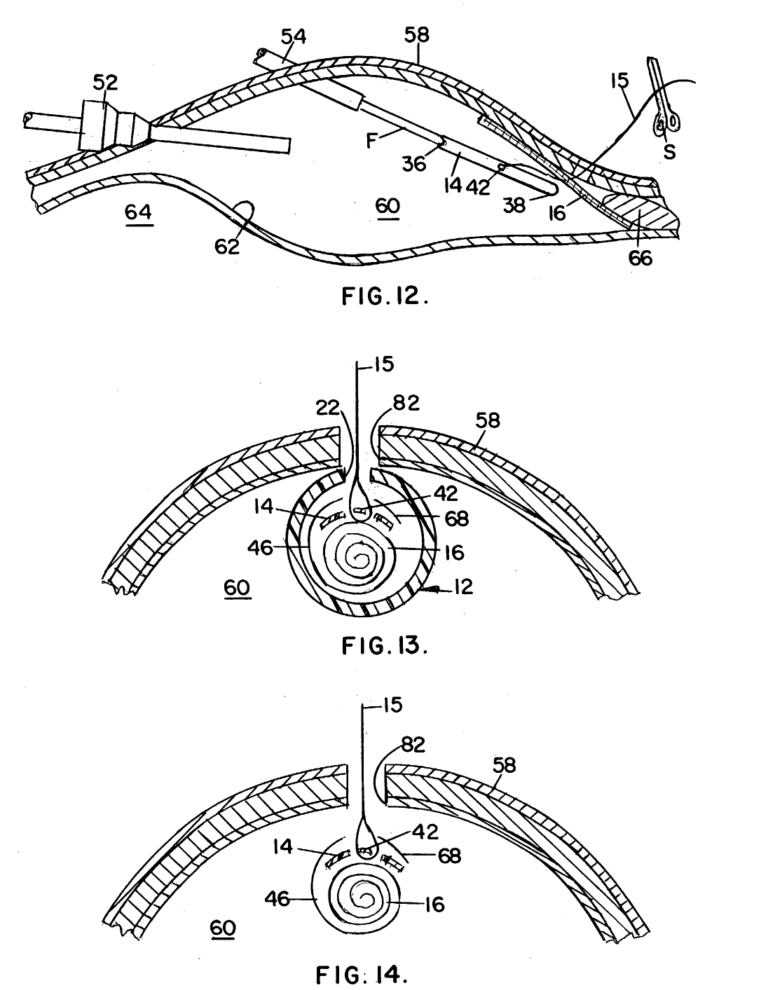

Once the mesh has been placed and stapled along its medial vertical edge, as shown in FIG. 11, anchor 14, or 114, or 214 is removed, as seen in FIG. 12. Forceps, not shown, are introduced through middle trocar 54, under direct vision, and the curved edge 36 of the anchor is grasped and pulled out of pre-peritoneal space 60 through middle trocar 54 bringing along string 15. Alternatively, string 15 can also be cut with laparoscopic scissors S and pulled out through supra-pubic trocar 56.

Once the anchor has been removed, the surgeon can resume unrolling and placing the mesh with bi-manual control. Two laparoscopic forceps, not shown, are re-introduced through middle trocar 54 and supra-pubic trocar 56. Circular orifice 48 of mesh 16 will allow insertion of the supra-pubic trocar into the pre-peritoneal space through the mesh.

It should be noted that mesh orifice 48 is much larger than the 5 mm trocar orifice. This will provide two advantages:

1. Just before placing the first staples on the medial vertical side of the mesh, the surgeon has some “wiggle room”, loosening the anchor and making some final adjustment to the mesh before stapling; and

2. The larger size mesh orifice facilitates re-insertion of the suprapubic trocar after the initial stapling of the mesh. The mesh orifice can be large, without compromising the effectiveness of the system.

The surgeon now removes trocars 52, 54 and 56 from the abdomen; pre-peritoneal space 60 is deflated and the wounds are closed.

FIG. 13 is a transverse cross-sectional showing of mesh anchor device 10 taken at the level of the supra-pubic trocar site, with the lower end of string 15 looped around neck 44 of tab 42 of anchor 14 and with the upper free end of the string passing through a supra-pubic trocar site orifice 82 and being disposed outwardly of abdomen 58.

FIG. 14 is a transverse cross-sectional view similar to FIG. 12 taken at the level of the supra-pubic trocar site following removal of sleeve 12, with outer convolution 46 of mesh roll 16 disposed between anchor 14 and abdominal wall 58, with string 15 passing through mesh orifice 48 and supra-pubic trocar orifice 82 and the loop of string 15 encircling neck 44 of tab 42 of anchor 14.

I claim:

1. A percutaneous mesh anchoring and placement device for use in groin hernia repair comprising: a tubular sleeve, a spirally-wound roll of prosthetic mesh releasably encased by the sleeve, an anchor releasably secured to the mesh and, a string attached to the anchor and extending through the mesh outwardly from the sleeve.

2. A percutaneous mesh anchoring and placement device according to claim 1, wherein the tubular sleeve has a closed end and an open end, the open end having an inwardly extending slot therein, with the string passing freely through the slot.

3. A percutaneous mesh anchoring and placement device according to claim 1, wherein the tubular sleeve has a closed end of a rounded, semi-circular configuration.

4. A percutaneous mesh anchoring and placement device according to claim 1, wherein the tubular sleeve has an open end of tapered, curvilinear configuration.

5. A percutaneous mesh anchor and placement device according to claim 1, wherin the tubular sleeve is fabricated from polycarbonate.

6. A percutaneous mesh anchoring and placement device according to claim 1, wherein the anchor has a tab to which the string is attached.

7. A percutaneous mesh anchoring and placement device according to claim 1, wherein the anchor has a pair of openings through which the string is threaded.

8. A percutaneous mesh anchoring and placement device according to claim 1, wherein the anchor has a pair of angularized slots through which the string is threaded.

9. A percutaneous mesh anchor and placement device according to claim 1, wherein the anchor is fabricated from polycarbonate.

10. A percutaneous mesh anchoring and placement device according to claim 1, wherein the anchor is positioned immediately below an outer convolution of the roll of mesh, the outer convolution being provided with an orifice for permitting passage of the string therethrough.

11. A percutaneous mesh anchor and placement device according to claim 10, wherein the orifice for permitting passage of the string therethrough will also permit the passage of a trocar therethrough.

12. A percutaneous mesh anchoring and placement device according to claim 1, wherein one of the convolutions of the roll of mesh has a lateral slit for accommodating bodily structures adjacent the hernia site.

13. A percutaneous mesh anchoring and placement device according to claim 1, wherein the sleeve is removable from the roll of surgical mesh.

14. A percutaneous mesh anchoring and placement device according to claim 1, wherein the anchor is removable from the roll of surgical mesh.

15. A percutaneous mesh anchoring and placement device according to claim 1, wherein the anchor is of lesser width and length than the width and length of the sleeve and the mesh.

16. A percutaneous mesh anchoring and placement device according to claim 1, wherein the mesh is of lesser length than the length of the sleeve.

17. A percutaneous mesh anchoring and placement device for use in groin hernia repair wherein trocars are positioned along a patient's abdominal midline and extend into an insufflated pre-peritoneal space, the device being introduced into the pre-peritoneal space through a selected one of the trocars and comprising: a tubular sleeve which encases a spirally-wound roll of prosthetic mesh having an anchor captured therein, the sleeve having a closed end and an open end, the anchor having a string attached thereto and extending outwardly from the open end of the sleeve, whereby the string may be grasped by the surgeon to position the device firmly against the patient's posterior abdominal wall adjacent the hernia, the mesh being separable from the sleeve, and the sleeve being removable from the pre-peritoneal space through a selected one of the trocars, whereupon the prosthetic mesh is partially unfurled and a portion thereof is stapled to the abdominal wall and the anchor is separated from the mesh and removed from the pre-peritoneal space through a selected one of the trocars and the prosthetic mesh is fully unfurled and its remaining edge portions are stapled to the abdominal wall to repair the hernia.

18. A percutaneous mesh anchoring and placement device according to claim 17, wherein the spirally wound roll of prosthetic mesh has an orifice therein for permitting the passage of the string and selected ones of the trocars therethrough.

19. In a percutaneous method of repairing a groin hernia using laparoscopic surgical procedures wherein an incision is made in a patient's abdomen and a patch is delivered via a trocar to the site of a hernia to be repaired, the improvement which comprises: inserting the patch into the abdomen as a rolled up sheet of a prosthetic mesh maintained in a rolled up form by a tubular, cylindrical sleeve, the mesh roll containing an anchor having a string attached thereto, the string having a free end which extends through provided orifices in the mesh and the sleeve, grasping the string by forceps to move the patch into overlaying relation to the hernia, maintaining tension on the string to hold the sleeve, anchor and mesh patch tightly against the abdominal wall adjacent the hernia, removing the sleeve, partially unfurling the mesh roll, and stapling the patch along one of its' edges to tissue adjacent the hernia, all while maintaining tension on the string, and removing the anchor and attached string, fully unfurling the mesh roll, and stapling the remaining edges of the patch to tissue adjacent the hernia.

20. In a percutaneous method of repairing a groin hernia according to claim 19, wherein the provided opening in the mesh is of sufficient size as to permit the passage of the trocar therethrough.
